(19) United States
(12) Patent Application Publication
(10) Pub. No.: US 2016/0367960 A1
(43) Pub. Date: Dec. 22, 2016

(54) METHOD AND SYSTEM FOR CARRYING OUT AN EXOTHERMIC GAS PHASE REACTION ON A HETEROGENEOUS PARTICULATE CATALYST

(71) Applicant: BASF SE, Ludwigshafen (DE)

(72) Inventors: Gerhard OLBERT, Dossenheim (DE); Gauthier Luc Maurice AVERLANT, Darmstadt (DE)

(73) Assignee: BASF SE, Ludwigshafen (DE)

(21) Appl. No.: 14/898,788

(22) PCT Filed: Jun. 16, 2014
(86) PCT No.: PCT/EP2014/062508

§ 371 Date: Dec. 16, 2015

(30) Foreign Application Priority Data
Jun. 17, 2013 (EP) 13172320.7

(51) Int. Cl. B01J 8/06; B01J 8/04; C01B 7/04; C10G 2/00; C07C 5/48

(52) U.S. Cl. CPC B01J 8/067; B01J 8/065; C10G 2/341; C07C 5/48; C01B 7/04; B01J 8/0496; B01J 2208/00212; B01J 2208/0053; B01J 2208/065

(57) ABSTRACT

The invention relates to a method for producing 1,3 butadien by means of the oxidative dehydration of n-butenes on a heterogenous particulate multimetal oxide catalyst which contains molybdenum as the active compound and at least one other metal and which is filled into the contact tubes (KR) of two or more tube bundle reactors (R-I, R-II), wherein a heat transfer medium flows around the intermediate space between the contact tubes (KR) of the two or more tube bundle reactors (R-I, R-II). The method includes a production mode and a regeneration mode which are carried out in an alternating manner. In the production mode, an n-butene-containing feed flow is mixed with an oxygen-containing gas flow and conducted as a supply flow (1) over the heterogenous particulate multimetal oxide catalyst filled into the contact tubes (KR) of the two or more tube bundle reactors (R-I, R-II), and the heat transfer medium absorbs the released reaction heat, minus the heat quantity used to heat the supply flow (1) to the reaction temperature in the production mode, by means of an indirect heat exchange and completely or partly dispenses the reaction heat onto a secondary heat transfer medium (H2Oliq) in an external cooler (SBK). In the regeneration mode, the heterogenous particulate multimetal oxide catalyst is regenerated by conducting an oxygen-containing gas mixture (3) over the catalyst and burning off the deposits accumulated on the heterogenous particulate multimetal oxide catalyst. The invention is characterized in that the two or more tube bundle reactors (R-I, R-II) have a single heat transfer medium circuit and as many of the two or more tube bundle reactors (R-I, R-II) as necessary are operated constantly in the production mode so that the released reaction heat, minus the heat quantity used to heat the supply flow (1) to the reaction temperature in the production mode, suffices to keep the temperature of the heat transfer medium in the intermediate spaces between the content tubes (KR) of all the tube bundle reactors (R-I, R-II) at a constant level with a variation range of maximally +/−10 DEG C.

### CROSS-REFERENCE TO RELATED APPLICATIONS

This application is a national stage application (under 35 U.S.C. §371) of PCT/EP2014/062508, filed Jun. 16, 2014, which claims benefit of European Application No. 13172320.7, filed Jun. 17, 2013, both of which are incorporated herein by reference in their entirety.

### DESCRIPTION

The invention relates to a process for carrying out an exothermic gas-phase reaction over a heterogeneous particulate catalyst.

As the reaction time increases, there are disadvantageous changes in the catalyst resulting in a decrease in catalyst activity.

Such changes can be, in particular, carbonization of the catalyst by deposits, as a result of which the number of active sites on the catalyst surface decreases. In other cases, the catalyst activity can decrease as a result of sintering of the catalyst.

Catalysts which are exposed to discontinuous, fluctuating feed streams which comprise impurities, for example comprising rare earths, are particularly prone to disadvantageous changes as the reaction time increases.

It is therefore necessary to regenerate the catalyst at regular intervals in order to restore the original activity as far as possible or as completely as possible.

For this purpose, it is generally necessary to ensure a sufficiently high temperature during regeneration for the operating life not to be impaired to an excessive degree after shutting down the reactor for the regeneration.

It was therefore an object of the invention to provide a process for carrying out heterogeneously catalyzed, exothermic gas-phase reactions, in which the regeneration of the catalyst can be carried out in a simple way without a deterioration in the operating life.

The object is achieved by a process for carrying out an exothermic gas-phase reaction over a heterogeneous particulate catalyst which has been introduced into the catalyst tubes of two or more shell-and-tube reactors, into the gap between the thermoplates of two or more thermoplate reactors or into the beds of two or more bed reactors through which heat exchanger devices run, where a heat transfer medium circulates through the intermediate space between the catalyst tubes of the two or more shell-and-tube reactors, through the thermoplates of the two or more thermoplate reactors or through the heat exchanger devices of the two or more bed reactors,

and the process comprises a production mode and a regeneration mode,

in the production mode, a gaseous feed stream is passed over the heterogeneous particulate catalyst and the heat transfer medium takes up, by indirect heat exchange, the heat of reaction liberated minus the quantity of heat consumed for heating the feed stream to the reaction temperature in all reactors in the production mode and releases all or part of it in an external apparatus and,

in the regeneration mode, the heterogeneous particulate catalyst is regenerated by passing a regeneration gas mixture over the catalyst, wherein
the two or more shell-and-tube reactors, thermoplate reactors or bed reactors have a single heat transfer medium circuit and
the number of the two or more shell-and-tube reactors, thermoplate reactors or bed reactors which are operated in the production mode is always such that the heat of reaction liberated minus the quantity of heat consumed for heating the feed stream to the reaction temperature in the production mode is sufficient for the temperature of the heat transfer medium in the intermediate spaces between the catalyst tubes of all shell-and-tube reactors, in the thermoplates of all thermoplate reactors or in the heat exchanger devices of the bed reactors to be kept constant with a fluctuation range of not more than +/−10° C.

It has been found that it is possible to regenerate heterogeneous, particulate catalysts in a simple way at the elevated temperatures essential to obtain activity and selectivity of said catalysts without external heaters being necessary for this purpose. The process of the invention also makes it unnecessary to reheat the reactor after regeneration to the reaction temperature for the production mode, for which there has hitherto been no reliable technical solution: electric heaters as have been used hitherto are unsuitable for frequent changes in operation in large-scale reactors; they tend, particularly because of the high proportion of ceramic materials, to suffer from damage and malfunctions and are also expensive to operate.

In particular, the process of the invention also ensures a continuous input stream for downstream process stages, with load fluctuations in a range of not more than about 50 to 120% compared to the nominal capacity.

The invention is not restricted in respect of the specific chemical reaction and can be employed for any exothermic gas-phase reaction which is carried out in the presence of heterogeneous catalysis. The process can particularly advantageously be applied to gas-phase oxidations of hydrocarbons, advantageously to the oxydehydrogenation of butenes to butadiene and to the methanation of CO or CO₂ by means of H₂ to form methane.

In a further embodiment, the process relates to reactions of gaseous feed streams which do not comprise any hydrocarbons. In particular, the process can be employed in the Deacon process, in oxidations, e.g. of hydrogen chloride to chlorine, in which redispersion of the active sites is necessary in order to counter sintering.

### A BRIEF DESCRIPTION OF THE FIGURES

FIG. 1 is a preferred way of carrying out the process according to the invention (2-reactor design), with only the plant parts relevant to the path of the gas streams both in the production mode and in the regeneration mode being shown in FIG. 1, and

FIGS. 2A, 2B, 2C is schematic depictions of a preferred way of carrying out the process of the invention (2-reactor design), with the parts of the plant relevant to the path of the heat transfer medium being shown.

The heterogeneous particulate catalyst can be an all-active catalyst or a coated catalyst. If it is a coated catalyst, it has a ceramic or zeolite-containing support which is enveloped by a shell comprising an active composition. Impregnated catalysts can likewise be used.

The invention is not subject to any restrictions in respect of the specific heterogeneous particulate catalyst; this can have any shape, e.g. rings, pellets, spheres, stars or monoliths.

The heterogeneous particulate catalyst is introduced to the catalyst tubes of two or more shell-and-tube reactors, into the gap between the thermoplates of two or more thermoplate reactors or into the beds of two or more bed reactors through which heat exchanger devices run.

The heat exchanger devices which run through the bed reactors are in particular tubes.

To remove the heat of reaction, use is made of a heat transfer medium which circulates through the intermediate space between the catalyst tubes of the two or more shell-and-tube reactors, through the thermoplates of the two or more thermoplate reactors or through the heat exchanger devices of the two or more bed reactors.

The process comprises a production mode and a regeneration mode.

In the production mode, a gaseous feed stream is passed over the heterogeneous particulate catalyst, resulting in an exothermic gas-phase reaction taking place, and the heat transfer medium takes up, by indirect heat exchange, the heat of reaction liberated minus the quantity of heat consumed in order to ensure heating of the feed stream to the reaction temperature in all reactors and releases part or all of this heat in an external apparatus.

The heat transfer medium can be any conventional liquid heat transfer medium, for example a salt melt, in particular a salt melt comprising potassium nitrate, potassium nitrite, sodium nitrite and/or sodium nitrate or a melt of metals such as sodium, mercury or alloys of various metals. It is also possible to use ionic liquids or heat transfer oils.

The heat transfer medium is in particular a salt melt and the external cooler is a salt bath cooler.

The secondary heat transfer medium is advantageously water which partly or completely vaporizes in the salt bath cooler. Steam generation is additionally achieved by means of this way of carrying out the process.

The gaseous feed stream is generally fed to the reactors at a temperature which is below the reaction temperature in order to avoid premature reactions and disadvantages associated therewith (decompositions, deposits, etc.). The reaction temperature should in general be reached only when the stream comes into contact with the heterogeneous particulate catalyst.

For this purpose, it is necessary to heat the feed stream, which is effected by using part of the heat of reaction which is liberated and taken up by the heat transfer medium.

The remaining heat of reaction taken up by the heat transfer medium is partly or completely released in an external apparatus. This can be a heat exchanger (cooler) but can also be a further reactor.

As soon as the activity of the heterogeneous particulate catalyst goes below a particular, prescribed value, operation is switched over from the production mode to the regeneration mode. The point in time at which the catalyst activity goes below the limit value for the decrease in catalyst activity is determined, in particular, by the decrease in conversion. Such a limit value for the decrease in conversion at which the switchover from the production mode to the regeneration mode is effected can be set differently depending on the specific reaction carried out. In particular, the above limit value can be set at a decrease in conversion of 25% at constant temperature. The increase in the pressure drop over the catalyst tubes as time goes on can also make regeneration necessary.

In the regeneration mode, the heterogeneous particulate catalyst is regenerated by passing a regeneration gas mixture over it.

Depending on the specific gas-phase reaction carried out in the production mode, this gas can be an oxygen-comprising gas or a reducing gas.

The regeneration mode comprises, in particular, the following regeneration steps:
flushing the catalyst tubes comprising the multimetal oxide catalyst with inert gas, in particular nitrogen, and
passage of a regeneration gas through the catalyst tubes comprising the multimetal oxide catalyst.

The flushing with inert gas is generally carried out by flushing the reactor a number of times using a total volume of inert gas corresponding to from three to five times the reactor volume, with the flushing gas being discharged in each case. At the end of the flushing phase, operation is generally switched over to circulation of the inert gas and the actual regeneration step is started by introduction of the regeneration gas being commenced.

The present invention has, in particular, the advantages of flexible operation which allows various sequences, for example a combination of flushing, burning-off, redispersion, reduction and/or reoxidation, since all the above processes proceed at similar temperature levels.

According to the invention, the two or more shell-and-tube reactors, thermoplate reactors or bed reactors have a single heat transfer medium circuit.

As soon as one of the shell-and-tube, thermoplate or bed reactors has to be switched over to the regeneration mode as a result of the prescribed limit value for the decrease in catalyst activity being reached, the connection to a single heat transfer medium circuit ensures that the temperature of the heat transfer medium does not drop but instead remains at a similar level compared to the reactors which continue to be operated in the production mode as a result of the heat liberated in the other reactors which continue to operate in the production mode also being supplied to the reactor in which the regeneration mode takes place.

According to the invention, the number of the shell-and-tube reactors, thermoplate reactors or bed reactors which are operated in the production mode is always such that the heat of reaction liberated minus the quantity of heat consumed for heating the feed stream to the reaction temperature in the production mode is sufficient for the temperature of the heat transfer medium in the intermediate spaces between the catalyst tubes of all shell-and-tube reactors, in the thermoplates of all thermoplate reactors or in the heat exchanger devices of all bed reactors to be kept constant with a fluctuation range of not more than +/−10° C.

In particular, from 30 to 90%, preferably from 50 to 80%, of the heat of reaction liberated minus the quantity of heat consumed for heating the feed stream to the reaction temperature in the production mode is utilized to keep the temperature of the heat transfer medium in the intermediate spaces between the catalyst tubes of all shell-and-tube reactors, thermoplate reactors or bed reactors constant with a fluctuation range of not more than +/−10° C.

The process is, in particular, carried out continuously.

In a preferred embodiment, two shell-and-tube reactors, thermoplate reactors or bed reactors are used.

In a further embodiment, from three to five shell-and-tube reactors, thermoplate reactors or bed reactors are used.

It is advantageous for all shell-and-tube reactors, thermoplate reactors or bed reactors to have the same capacity in respect of the desired product.

In a further embodiment, the capacity in respect of the desired product of the two or more shell-and-tube reactors, thermoplate reactors or bed reactors differs by from −30 to +30%, preferably from −10 to +20%.

The temperature of the heat transfer medium in the intermediate space between the catalyst tubes of the two or more shell-and-tube reactors, in the thermoplates of all thermoplate reactors or in the heat exchanger devices of all bed reactors is, in particular, kept constant at a value in the range from 200 to 600° C., preferably at a value in the range from 350 to 450° C., particularly preferably at a value in the range from 380 to 420° C.

Depending on the specific type of change in the catalyst activity, for example as a result of deposits of foreign materials, it can be advantageous to convey the regeneration gas mixture through the appropriate reactor in the regeneration mode in the opposite direction compared to the reaction gas in the production mode.

The invention also provides a plant for carrying out the above process, comprising two shell-and-tube reactors each having a plurality of catalyst tubes into which a heterogeneous particulate catalyst has been introduced,

and comprising in each case an upper and a lower ring line at the upper and lower end, respectively, of each shell-and-tube reactor, which are connected to the intermediate spaces between the catalyst tubes and in which a heat transfer medium circulates in each case with the aid of a pump,

where the lower ring line of each of the shell-and-tube reactors is connected to the upper ring line of the other shell-and-tube reactor via a connecting line which can in each case be closed or partly or fully opened by means of a shut-off device and to an open equalization line which is physically separate from the connecting lines and connects the upper ring lines,

and also comprising an external cooler which is connected to each of the lower ring lines in each case via an input line which can be regulated in each case by means of a sliding valve and is connected in each case to the upper ring line by means of a discharge line.

The invention also provides a plant for carrying out the above process, comprising two shell-and-tube reactors having parallel longitudinal axes and in each case having a plurality of catalyst tubes into which a heterogeneous particulate catalyst has been introduced,

comprising an intermediate chamber between the two shell-and-tube reactors which is open to the intermediate spaces between the catalyst tubes of the shell-and-tube reactors as a result of openings being provided in the mutually opposite subregions of the reactor shell of the shell-and-tube reactors and

is closed to the outside by means of two longitudinal walls and also an upper cover and a lower cover,

comprising three or more deflection plates which are alternately configured as deflection plates which extend over the cross section of both reactors and the intermediate chamber and leave passages free in the outer regions facing away from one another of the two reactors or are configured as two disk-shaped deflection plates which extend completely over the cross section of each reactor but leave the region of the intermediate chamber open,

where the shell-and-tube reactors are free of catalyst tubes in the deflection regions of the deflection plates,

where the intermediate chamber is connected to an external cooler

and a heat transfer medium is conveyed by means of a pump through the intermediate space between the catalyst tubes of the shell-and-tube reactors, through the intermediate chamber and through the external cooler.

The invention is illustrated below with the aid of a drawing.

The drawing shows in detail:

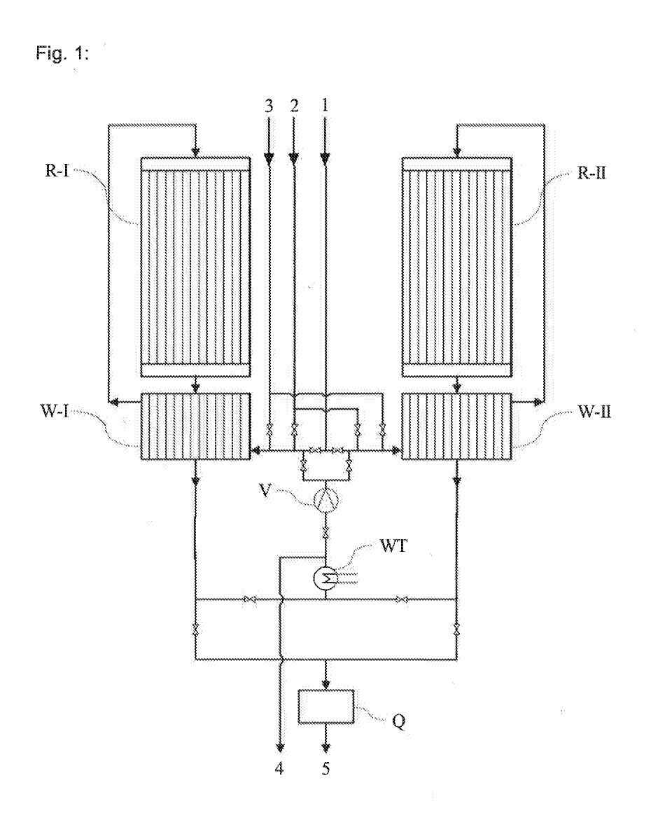

FIG. 1 a preferred way of carrying out the process according to the invention (2-reactor design), with only the plant parts relevant to the path of the gas streams both in the production mode and in the regeneration mode being shown in FIG. 1, and

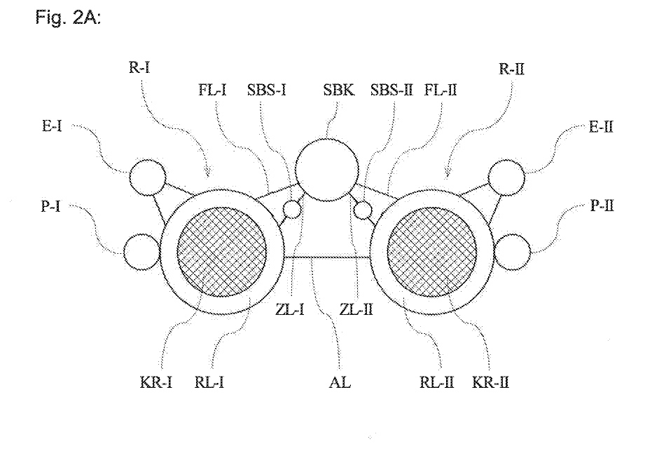

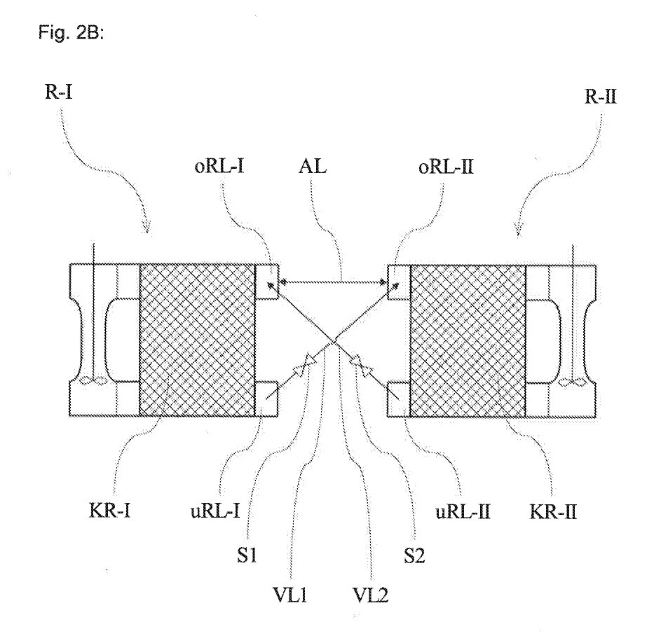

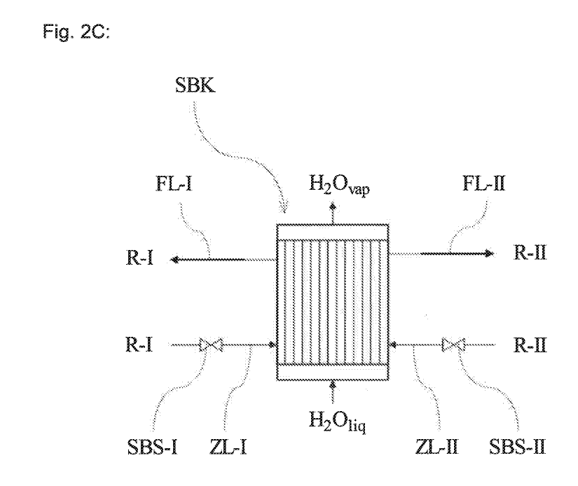

FIGS. 2A, 2B, 2C schematic depictions of a preferred way of carrying out the process of the invention (2-reactor design), with the parts of the plant relevant to the path of the heat transfer medium being shown.

The schematic depiction in FIG. 1 shows a preferred embodiment according to the invention (2-reactor design), with only the path of the gas streams but not of the heat transfer medium being shown in the figure:

In the reaction mode, both shell-and-tube reactors R-I, R-II are supplied in the upper region of said reactors with a feed stream 1 which is preheated beforehand by means of a cross-current heat exchanger W in each case using the product gas mixture leaving the respective shell-and-tube reactor R-I, R-II. The product gas mixture flows from the lower region of each of the shell-and-tube reactors R-I, R-II, heats the input stream in the cross-current heat exchanger W and is subsequently cooled in a quench Q. In the preferred embodiment depicted in FIG. 1, the two streams leaving the cross-current heat exchangers W are combined before being introduced into the quench Q.

As soon as the catalyst activity in one of the shell-and-tube reactors R-I, R-II, for example in the shell-and-tube reactor R-II, has dropped below a prescribed value, this reactor is switched over from the reaction mode to the regeneration mode while the other reactor, in the present embodiment reactor R-I, continues to be operated in the reaction mode. For this purpose, stream 1 is still fed into the reactor R-I but not into the reactor R-II which is, in contrast, firstly flushed with inert gas, in particular nitrogen, stream 2. Stream 2 is passed through the cross-current heat exchanger W and from the top downward through the catalyst tubes KR of the shell-and-tube reactor R and subsequently discharged via line 4, with flushing being carried out a number of times until from three to five times the reactor volume has been replaced. At the end of the flushing phase, stream 2 can also be circulated via the additional heat exchanger WT and the compressor V.

The flushing phase of the regeneration mode is followed by the actual regeneration phase in which regeneration gas, in particular air, particularly preferably lean air, stream 3, is introduced. Stream 3 is likewise conveyed via the cross-current heat exchanger W from the top downward through the catalyst tubes KR of the shell-and-tube reactor R, but subsequently circulated via an additional heat exchanger WT and a compressor V. It is also possible to use a further quench Q instead of the additional heat exchanger WT.

FIGS. 2A to 2C show, in contrast, the path of the heat transfer medium for the same embodiment according to the invention (2-reactor design) shown in FIG. 1 for the path of the gas streams:

The cross section depicted in FIG. 2A shows the two shell-and-tube reactors R-I, R-II, with schematically indicated sections through the catalyst tubes KR, and also ring lines RL for the heat transfer medium. An electric heater E-I, E-II is provided for each of the two shell-and-tube reactors R-I, R-II. The heat transfer medium is conveyed via a pump P-I, P-II in each case. The ring lines RL are each connected to a salt bath cooler SBK via in each case an input line ZL-I, ZL-II, which is regulated by means of salt bath slide valves SBS-I, SBS-II, and via discharge lines FL-I, FL-II. An equalization line AL is provided between the ring lines RL of the two shell-and-tube reactors R-I, R-II.

The longitudinal section depicted in FIG. 2B shows the connection of the lower ring line uRL-I of the shell-and-tube reactor R-I to the upper ring line oRL-II of the second shell-and-tube reactor R-II via a connecting line VL having a connecting slide valve S1 or of the lower ring line uRL-II of the second shell-and-tube reactor R-II to the upper ring line oRL-I of the first shell-and-tube reactor R-I via a connecting line VL having a connecting slide valve S2. The pressure and suction sides for the flow of the heat transfer medium are denoted by p+ and p−, respectively. The two upper ring lines oRL-I, oRL-II are connected via an open equalization line AL.

FIG. 2C schematically shows a longitudinal section through the salt bath cooler SBK, which is configured by way of example as a shell-and-tube heat exchanger, having input lines ZL-I, ZL-II regulated by means of salt bath slide valves SBS-I, SBS-II from the shell-and-tube reactors R-I, R-II and discharge lines FL-I, FL-II at the opposite end of the salt bath cooler SBK. As secondary heat transfer medium, use is made, by way of example, of water which forms steam in the salt bath cooler SBK.

1.-19. (canceled)

20. A process for carrying out an exothermic gas-phase reaction over a heterogeneous particulate catalyst which has been introduced into the catalyst tubes of two or more shell-and-tube reactors (R-I, R-II), into the gap between the thermoplates of two or more thermoplate reactors or into the beds of two or more bed reactors through which heat exchanger devices run, where a heat transfer medium circulates through the intermediate space between the catalyst tubes (KR) of the two or more shell-and-tube reactors (R-I, R-II), through the thermoplates of the two or more thermoplate reactors or through the heat exchanger devices of the two or more bed reactors,
and the process comprises a production mode and a regeneration mode,
in the production mode, a gaseous feed stream (1) is passed over the heterogeneous particulate catalyst and the heat transfer medium takes up, by indirect heat exchange, the heat of reaction liberated minus the quantity of heat consumed for heating the feed stream (1) to the reaction temperature in the production mode and releases all or part of it in an external apparatus and,
in the regeneration mode, the heterogeneous particulate catalyst is regenerated by passing a regeneration gas mixture (3) over the catalyst, wherein
the two or more shell-and-tube reactors (R-I, R-II), thermoplate reactors or bed reactors have a single heat transfer medium circuit and
the number of the two or more shell-and-tube reactors (R-I, R-II), thermoplate reactors or bed reactors which are operated in the production mode is always such that the heat of reaction liberated minus the quantity of heat consumed for heating the feed stream (1) to the reaction temperature in all shell-and-tube reactors (R-I, R-II), thermoplate reactors or bed reactors in the production mode is sufficient for the temperature of the heat transfer medium in the intermediate spaces between the catalyst tubes (KR) of all shell-and-tube reactors (R-I, R-II), in the thermoplates of all thermoplate reactors or in the heat exchanger devices of the bed reactors to be kept constant with a fluctuation range of not more than +/−10° C., where at least one of the two or more shell-and-tube reactors (R-I, R-II), thermoplate reactors or bed reactors is operated in the regeneration mode and the heat of reaction liberated in the remainder of the two or more shell-and-tube reactors (R-I, R-II), thermoplate reactors or bed reactors which are operated in the production mode minus the quantity of heat consumed for heating the feed stream (1) to the reaction temperature in the production mode is partly removed via the external cooler and the remainder is utilized to keep the temperature of the heat transfer medium in the intermediate spaces between the catalyst tubes (KR) of all shell-and-tube reactors (R-I, R-II), thermoplate reactors or bed reactors constant with a fluctuation range of not more than +/−10° C., so that the temperature of the heat transfer medium in the reactor in which the regeneration mode takes place also does not drop but instead remains at a similar level compared to the reactors which continue to be operated in the production mode.

21. The process according to claim 20, wherein the process is carried out continuously.

22. The process according to claim 20, wherein the heat transfer medium is a salt melt and the external cooler (SBK) is a salt bath cooler.

23. The process according to claim 22, wherein the secondary heat transfer medium (H₂O_{liq}) is water which partly or completely vaporizes in the salt bath cooler (SBK).

24. The process according to claim 23, wherein from 30 to 90% of the heat of reaction liberated minus the quantity of heat consumed for heating the feed stream (1) to the reaction temperature in all shell-and-tube reactors (R-I, R-II), thermoplate reactors or bed reactors in the production mode is utilized to keep the temperature of the heat transfer medium in the intermediate spaces between the catalyst tubes (KR) of all shell-and-tube reactors (R-I, R-II), thermoplate reactors or bed reactors constant with a fluctuation range of not more than +/−10° C.

25. The process according to claim 20, wherein the temperature of the heat transfer medium in the intermediate space between the tubes of all shell-and-tube reactors (R-I, R-II), in the thermoplates of all thermoplate reactors or in the heat exchanger devices of all bed reactors is kept constant with a fluctuation range of +/−5° C.

26. The process according to claim 20, wherein two shell-and-tube reactors (R-I, R-II), thermoplate reactors or bed reactors are used.

27. The process according to claim 20, wherein from 3 to 5 shell-and-tube reactors (R-I, R-II), thermoplate reactors or bed reactors are used.

28. The process according to claim 20, wherein all shell-and-tube reactors (R-I, R-II), thermoplate reactors or bed reactors have the same capacity in respect of the desired product.

29. The process according to claim 20, wherein the production capacity in respect of the desired product of the two or more shell-and-tube reactors (R-I, R-II), thermoplate reactors or bed reactors differs by −30 to +30%.

30. The process according to claim 20, wherein the regeneration mode comprises the following regeneration steps:
flushing the catalyst tubes comprising the multimetal oxide catalyst with inert gas and
passage of a regeneration gas through the catalyst tubes comprising the multimetal oxide catalyst.

31. The process according to claim 30, wherein the regeneration gas is an oxygen-comprising gas.

32. The process according to claim 30, wherein the regeneration gas is a reducing gas.

33. The process according to claim 30, wherein the passage of a regeneration gas is a reduction, burning-off and/or a redispersion of the active sites of the heterogeneous particulate catalyst.

34. The process according to claim 20, wherein the temperature of the heat transfer medium in the intermediate space between the catalyst tubes (KR) of the two or more shell-and-tube reactors (R-I, R-II), in the thermoplates of all thermoplate reactors or in the heat exchanger devices of all bed reactors is maintained at a value in the range from 200 to 600° C.

35. The process according to claim 20, wherein, in the regeneration mode, the regeneration gas mixture is conveyed through the respective reactor in the opposite direction compared to the reaction gas in the production mode.

36. A plant for carrying out the process according to claim 26,
Comprising two shell-and-tube reactors (R-I, R-II) each having a plurality of catalyst tubes (KR) into which a heterogeneous particulate catalyst has been introduced,
and comprising in each case an upper ring line (oRL-I, oRL-II) and a lower ring line (uRL-I, uRL-II) at the upper and lower end, respectively, of each shell-and-tube reactor (R-I, R-II), which are connected to the intermediate spaces between the catalyst tubes (KR) and in which a heat transfer medium circulates with the aid of a pump (P),
where the lower ring line (uRL-I, uRL-II) of each of the shell-and-tube reactors (R-I, R-II) is connected to the upper ring line (oRL-I, oRL-II) of the other shell-and-tube reactor (R-I, R-II) via a connecting line (VL) which can in each case be closed or partly or fully opened by means of a shut-off device (S1, S2) and to an open equalization line (AL) which is physically separate from the connecting lines (VL) and connects the upper ring lines (oRL-I, oRL-II),
and also comprising an external cooler (SBK) which is connected to each of the lower ring lines (uRL-I, uRL-II) in each case via an input line (ZL-I, ZL-II) which can be regulated in each case by means of a sliding valve (SBS-I, SBS-II) and is connected in each case to the upper ring line (oRL-I, oRL-II) by means of a discharge line (FL-I, FL-II).

37. A plant for carrying out the process according to claim 26, comprising two shell-and-tube reactors (R-I, R-II) having parallel longitudinal axes and in each case having a plurality of catalyst tubes (KR) into which a heterogeneous particulate catalyst has been introduced,
comprising an intermediate chamber between the two shell-and-tube reactors (R-I, R-II)
which is open to the intermediate spaces between the catalyst tubes (KR) of the shell-and-tube reactors (R-I, R-II) as a result of openings being provided in the mutually opposite subregions of the reactor shell of the shell-and-tube reactors (R-I, R-II) and
is closed to the outside by means of two longitudinal walls and also an upper cover and a lower cover,
comprising three or more deflection plates which are alternately configured as deflection plates which extend over the cross section of both reactors and the intermediate chamber and leave passages free in the outer regions facing away from one another of the two reactors (R-I, R-II) or are configured as two disk-shaped deflection plates which extend completely over the cross section of each reactor (R-I, R-II) but leave the region of the intermediate chamber open,
where the shell-and-tube reactors (R-I, R-II) are free of catalyst tubes (KR) in the deflection regions of the deflection plates,
where the intermediate chamber is connected to an external cooler (SBK)
and a heat transfer medium is conveyed by means of a pump (P) through the intermediate space between the catalyst tubes (KR) of the shell-and-tube reactors (R-I, R-II), through the intermediate chamber and through the external cooler (SBK).
